(19) United States
(12) Patent Application Publication
(10) Pub. No.: US 2016/0317172 A1
(43) Pub. Date: Nov. 3, 2016

(54) TISSUE EXCISION DEVICE

(71) Applicant: OLYMPUS CORPORATION, Tokyo (JP)

(72) Inventors: Yoshiyuki KUMADA, Tokyo (JP); Kayuri KIMURA, Saitama (JP)

(73) Assignee: OLYMPUS CORPORATION, Tokyo (JP)

(21) Appl. No.: 15/206,512

(22) Filed: Jul. 11, 2016

Related U.S. Application Data
(63) Continuation of application No. PCT/JP2015/056107, filed on Mar. 2, 2015.

(30) Foreign Application Priority Data
Mar. 13, 2014 (JP) 2014-050419

(51) Int. Cl. A61B 17/3205; A61B 17/3201

(52) U.S. Cl. CPC A61B 17/3205; A61B 17/3201

(57) ABSTRACT

This tissue excision device has the following elements: a jaw disposed at a distal end of an insertion portion and used to sandwich tissue; a cutter movable along a track formed along the shape of the jaw; a drive unit which generates force to drive the cutter; and a force transmission member which connects the drive unit and the cutter and accommodated in an accommodation portion provided along the track, and which transmits the force generated by the drive unit to the cutter, and the drive unit is configured to generate greater force as reaction force from the accommodation portion becomes greater.

### CROSS-REFERENCE TO RELATED APPLICATIONS

This application is a Continuation Application of International Application No. PCT/JP2015/056107 filed on Mar. 2, 2015, which claims priority to Japanese Application No. 2014-050419 filed on Mar. 13, 2014. The contents of International Application No. PCT/JP2015/056107 and Japanese application No. 2014-050419 are incorporated herein by reference in its entirety.

### TECHNICAL FIELD

The present invention relates to a tissue excision device.

### BACKGROUND ART

There are conventionally known suture instruments (for example, PTL 1) which have the following elements: a curved pair of jaws for sandwiching tissue, which can be opened and closed and provided at the tip of an inserted portion to be inserted in a body; a cutter movable along a track which is formed along the shape of the jaw and which is formed at a middle position of the jaw; and a staple shooting means which shoots staples toward the tissue sandwiched by the jaws at the both sides relative to the cutter.

In the inserted portion and the jaw, the suture instrument has a flexible shaft which deforms along the shape of the inserted portion and the jaw. Also, the flexible shaft pushes and moves the cutter along the track when the flexible shaft is pushed in the longitudinal direction at the proximal end thereof, and the staple is protruded from the contacting surface of the jaws along with the movement of the cutter, which makes it possible to cut tissue sandwiched by the jaws by the cutter and suture the both sides of the cut surface by the staples.

### CITATION LIST

### Patent Literature

{PTL 1} Japanese Unexamined Patent Application, Publication No. H08-289895

### SUMMARY OF INVENTION

An aspect of the present invention provides a tissue excision device comprising: a jaw which is disposed at a distal end of an insertion portion and which is used to sandwich tissue; a cutter which is movable along a track formed along the shape of the jaw; a drive unit which generates force to drive the cutter; and a force transmission member which connects the drive unit and the cutter and accommodated in an accommodation portion provided along the track, and which transmits the force generated by the drive unit to the cutter, wherein the drive unit is configured to generate greater force as reaction force that the force transmission member receives from the accommodation portion becomes greater.

### BRIEF DESCRIPTION OF DRAWINGS

FIG. 1 is a perspective view of a tissue excision device according to an embodiment of the present invention.

FIG. 2A is a cross-sectional view of a suturing portion of the tissue excision device shown in FIG. 1.

FIG. 2B is a cross-sectional view of the suturing portion of the tissue excision device shown in FIG. 1.

FIG. 3 is a schematic plan view of a cutter, a cutter slot, a wire, and a driving unit of the suturing portion shown in FIG. 2.

FIG. 4 is a view showing a model of FIG. 3.

FIG. 5 is a graph showing example torque generated by a motor which constitutes the drive unit (drive device) of FIG. 3.

FIG. 6A is a schematic plan view showing a first modified example of the suturing portion shown in FIG. 3.

FIG. 6B is a graph showing example torque generated by the motor of the first modified example of the suturing portion shown in FIG. 3.

FIG. 7A is a schematic plan view showing a second modified example of the suturing portion shown in FIG. 3.

FIG. 7B is a graph showing example torque generated by the motor of the second modified example of the suturing portion shown in FIG. 3.

FIG. 8A is a schematic plan view of a third modified example which shows a modified suturing portion of FIG. 3.

FIG. 8B is a schematic plan view of a fourth modified example which shows a modified suturing portion of FIG. 3.

FIG. 9A is a modified example of the drive unit shown in FIG. 3 and shows a schematic plan view of a suturing portion.

FIG. 9B is a modified example of the drive unit shown in FIG. 3 and shows a graph of tension generated by a spring which constitutes the driving unit.

### DESCRIPTION OF EMBODIMENTS

A tissue excision device 1 according to an embodiment of the present invention will be described below with reference to the drawings.

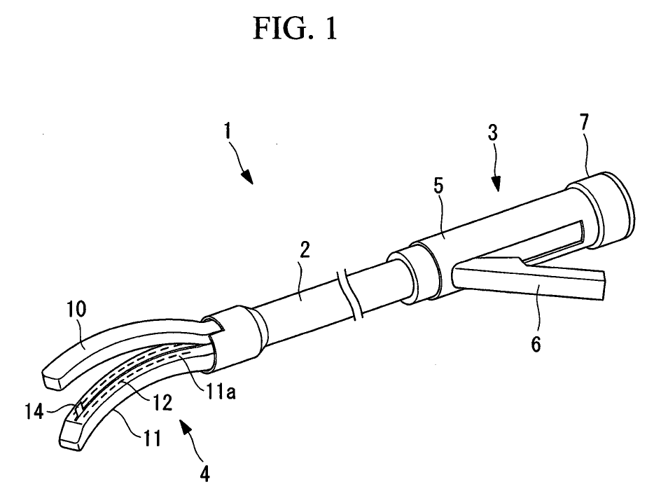

The tissue excision device 1 according to this embodiment is a suturing device, and has the following elements: an insertion portion (insertion tube) 2; operation portion 3 provided at the proximal end portion of the insertion portion 2; and a suturing section (jaws) 4 disposed at the tip of the insertion portion 2 as shown in FIG. 1.

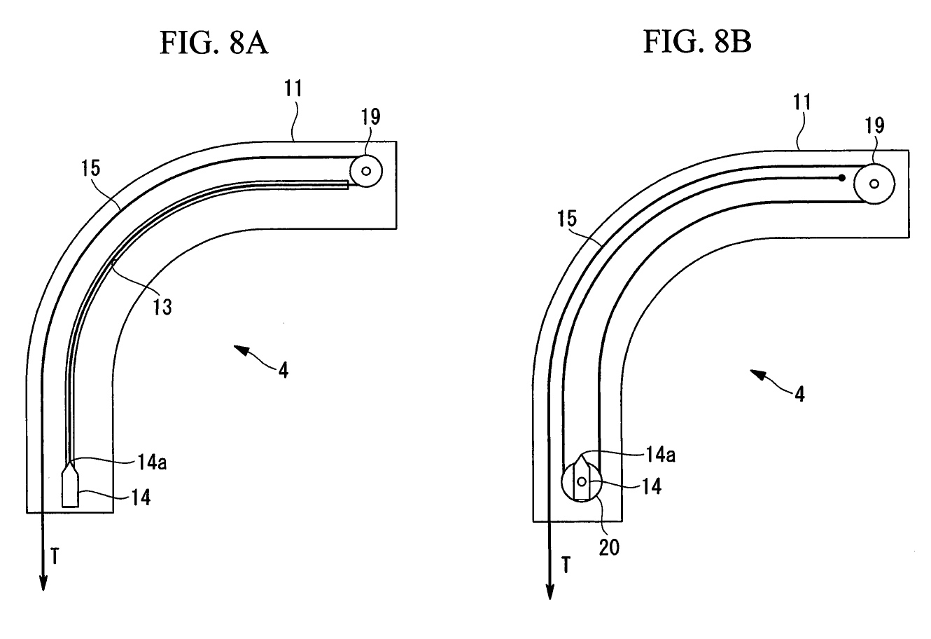

The operation portion 3 comprises a handle 5 which an operator manipulates, a lever 6 which is comprised with the handle 5 and is for shooting staples wherein the lever 6 is pivotable, and an anvil open/close knob 7. Also, the operation portion 3 accommodates a motor 8 (shown in FIG. 8) moved by operation of the lever 6 for shooting staples.

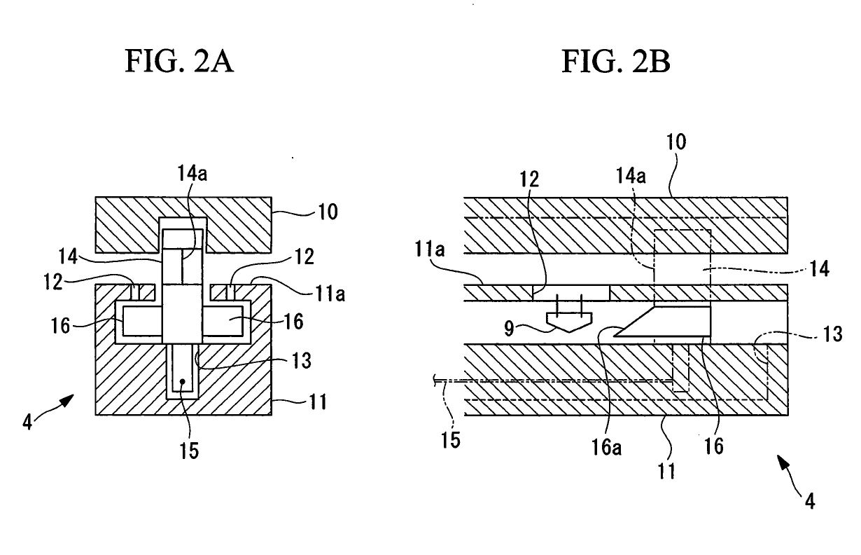

The suturing section 4 comprises an anvil 10 which receives the staple 9 (shown in FIG. 2B) and then bends the staple 9, and a cartridge 11 which accommodates a plurality of staples 9 and which releases the staples.

The anvil 10 and the cartridge 11 are consisting jaws which is bent toward the lateral direction relative to the longitudinal direction, and the anvil 10 pivots by operating the anvil open/close knob 7 provided in the handle 5. When the anvil 10 pivots to the adjacent position to the cartridge 11, the surface of the cartridge 11 which faces the anvil 10 and the surface of the anvil 10 which faces the cartridge 11 become substantially parallel, and the surfaces can grasp and hold tissue between them.

The surface 11a which faces the anvil 10 has a plurality of slots 12. The slots accommodate U-shaped staples 9 which are made of titanium alloy or stainless steel. So that the staples 9 can be protruded through the openings of the slots 12 in a state in which the leg tips are directed toward the surface 11a. The slots 12 are arranged along a cutter slot 13 described below.

A cutter slot 13 is located between the rows of the staples 9 in the cartridge 11, and the cutter slot 13 (accommodation portion) supports the cutter 14 so that the cutter 14 can slide along the longitudinal direction of the cartridge 11. The cutter slot 13 is located at the middle in the width of the cartridge 11 and also provided along the bended shape of the cartridge 11. As shown in FIGS. 2A and 2B, part of the cutter 14 is accommodated in the cutter slot 13, and a wire (force transmission member) 15 whose one end is connected to the cutter 14 is accommodated in the cutter slot 13.

Also, as shown in FIGS. 2A and 2B, a staple pushing member 16, which has an oblique surface 16a for pushing out the staples 9 through the slots 12, is fixed to the cutter 14. The staple pushing member 16 is located in a space provided in the cartridge 11 and inside the slot 12, and moves integrally with the cutter 14 when the cutter 14 is moved along the cutter slot 13, and then the oblique surface 16a pushes out the staples 9 from the slots 9 to the position which is above the surface 11a of the cartridge 11.

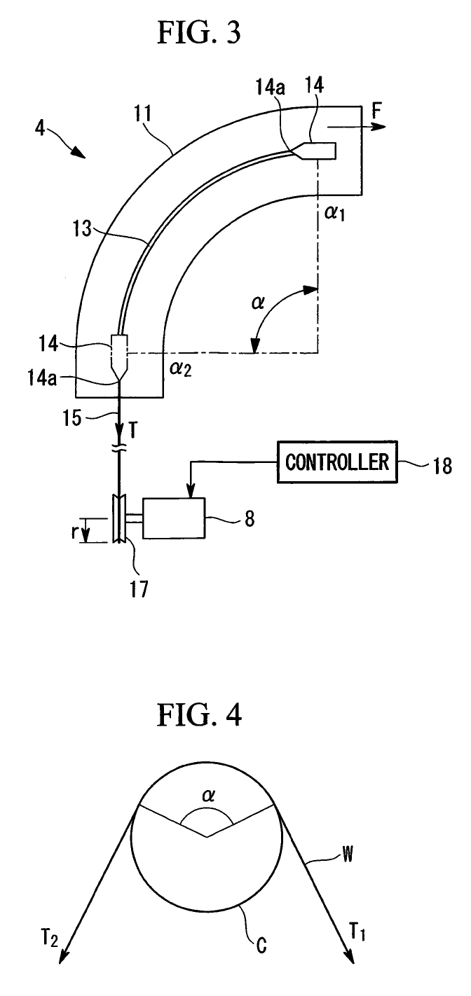

As shown in FIG. 3, at the initial state, the cutter 14 is positioned at a first position which is at the distal end side of the cartridge 11, and the tip 14a of the cutter 14 is directed to the proximal end of the cartridge 11 at the first position. Also, the oblique surface 16a of the staple pushing member 16 slants so that the distance from the surface 11a of the cartridge 11 becomes larger as it goes toward the proximal end.

The other end of the wire 15 is connected via the insertion portion 2 to a pulley 17 which is accommodated in the operation portion 3 so that the wire 15 can be wounded around the pulley 17. When the wire 15 is wounded by the pulley 17 driven by the motor 8, the cutter 14 and the staple pushing member 16, which are connected to the one end of the cutter 14, move along the cutter slot 13 to a second position which is at the proximal end side of the cartridge 11.

In this embodiment, a controller 18 which controls torque of the motor 8 is connected to the motor 8.

As shown in FIG. 3, in a case in which the cutter slot 13 of the cartridge 11 is curved in an arc shape, since the wire 15 accommodated in the cutter slot 13 has contact with the internal wall of the cutter slot 13, the cutter 14 moves against the friction force cased therebetween. In this case, a force relationship which is generated when a wire W is wounded around a cylinder C is generated.

Thus, when the contact angle between the wire W and the cylinder C is α, when the friction force coefficient between them is μ, and when the tension applied to the both sides of the wire W so that the cylinder C is located between the both sides of the wire W are T₁ and T₂, the following expression (1) is satisfied.

T₁=T₂×exp(−μα) (1)

In a case in which those elements are replaced with the relationship between the wire 15 and the internal wall of the cutter slit 13, when the tension applied to the wire 15 is T, the friction force coefficient between the wire 15 and the internal wall of the cutter slit 13 is μ, and the contact angle of them is α, the following expression (2) is satisfied.

T=T₀×exp(μα) (2)

In this expression, T₀ is thrust of the cutter 14 when α=0.

The thrust T₀ is a force needed to cut tissue while moving the cutter 14 along a straight track which causes no friction force, and the thrust T₀ can be empirically obtained.

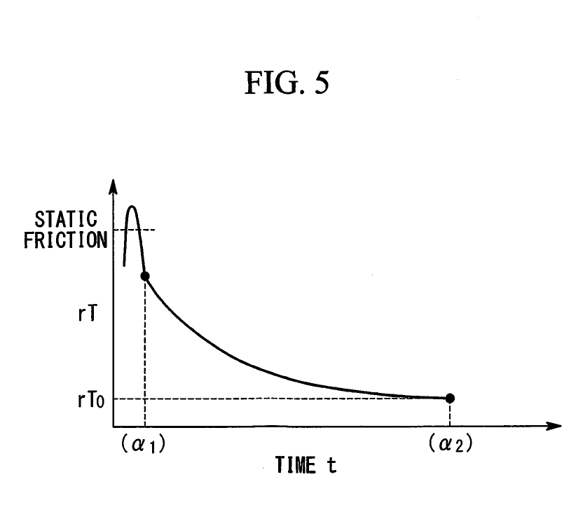

As shown in FIG. 2, the controller 18 controls the tension T so that a constant thrust T₀ of the cutter 14 is obtained even when the contact angle α varies. Specifically, when the wounding radius of the pulley 17 connected to the motor 8 is r, the controller 18 controls the motor 8 so that the torque rT shown FIG. 5 is generated in response to the time t from the initiation of the operation.

By this control, the controller 18 controls the motor 8 so that the torque rT becomes smaller as the contact angle α becomes smaller when the cutter 14 is moved and the contact angle α is changed from the contact angle α₁ between the wire 15 and the internal wall of the cutter slot 13 at the first position to the contact angle α₂ at the second position.

In FIG. 5, torque which exceeds the static friction is generated just after the initiation of the operation, and then the torque calculated by the expression (2) is generated.

An effect of the tissue excision device in accordance with this embodiment is described below.

When tissue of an in vivo is excised using the tissue excision device 1 in accordance with this embodiment, an operator holds and manipulates the handle 5, inserts the insertion portion 2 from its tip end side into the a body cavity or a space in the body, positions the suturing section 4 provided on the tip of the insertion portion 2 at a position adjacent to a portion to be excised.

Then tissue is positioned between the anvil 10 and the cartridge 11, and the anvil 10 is pivoted relative to the cartridge 11 and then the tissue is sandwiched between them by rotating the anvil open/close knob 7 of the handle 5.

In this state, when the operator pivots the lever 6 for shooting staples so that the lever 6 is retracted, the controller 18 makes the motor 8 operates, the cutter 14 and the staple pushing member 16 are made to slide along the cutter slit 13 from the distal end side to the proximal end side thereof by wounding the wire 15 around the pulley 17.

By the aforementioned operation, the tip 14a of the cutter 14 cuts the tissue sandwiched between the cartridge 11 and the anvil 10, and the staples 9 are pushed out from the slots 12 by the oblique surface 16a of the staple pushing member 16 and penetrate the tissue, and then the staples 9 are deformed by the anvil 10 and thereby sequentially suture the tissue.

In this state, according to the tissue excision device 1 of this embodiment, since the controller 18 controls the motor 8 so that the torque becomes greater as the contact angle α between the wire 15 accommodated in the cutter slot 13 and the internal wall of the cutter slot 13 becomes greater, the motor 8 is operated with large torque when the contact angle α and the friction are large, and the motor 8 is operated with small torque when the contact angle α and the friction are small. Therefore, variation of the speed of the cutter 14 depending on the positions can be reduced.

Consequently, variation of blood circulation state in the tissue to be excised may not occur, and variation of the degree of tissue healing after the surgery can be reduced. Thus, blood circulation states in the tissue in the respective excised portions vary when the speed of the cutter 14 varies, and therefore the speed of tissue healing also varies in each of the portions. However, the tissue excision device 1 according to this embodiment does not cause such inconveniences. Therefore, it is possible to move the cutter 14 in a low speed, waiting for recirculation of blood at the excised portions, and also it is possible to avoid a situation in which the position of the tissue sandwiched by the anvil 10 and the cartridge 11 is moved by preventing sudden increase of the speed of the cutter 14, which are advantages of this embodiments.

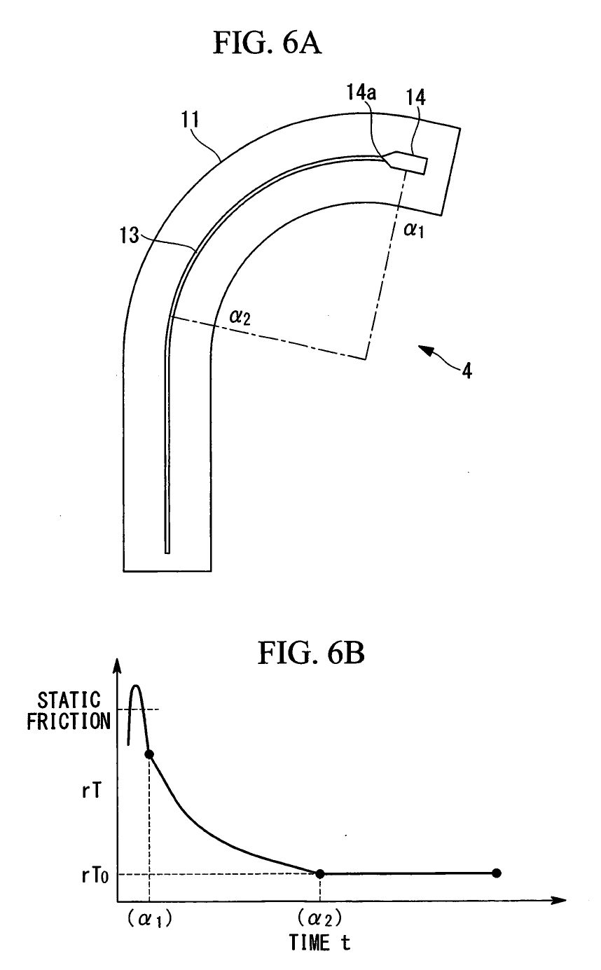

In this embodiment, the suturing section 4 which is simply arc-shaped and curved is described. In another embodiment, the aforementioned configuration can be applied to a suturing section 4 which has a shape by combining a curved portion and a straight portion as shown in FIG. 6A. In this configuration, since the straight portion is located at the proximal end side of the cutter slot 13, the motor 8 can be controlled to operate with a constant torque rT₀ as shown in FIG. 6B after the cutter 14 has been located in the straight portion.

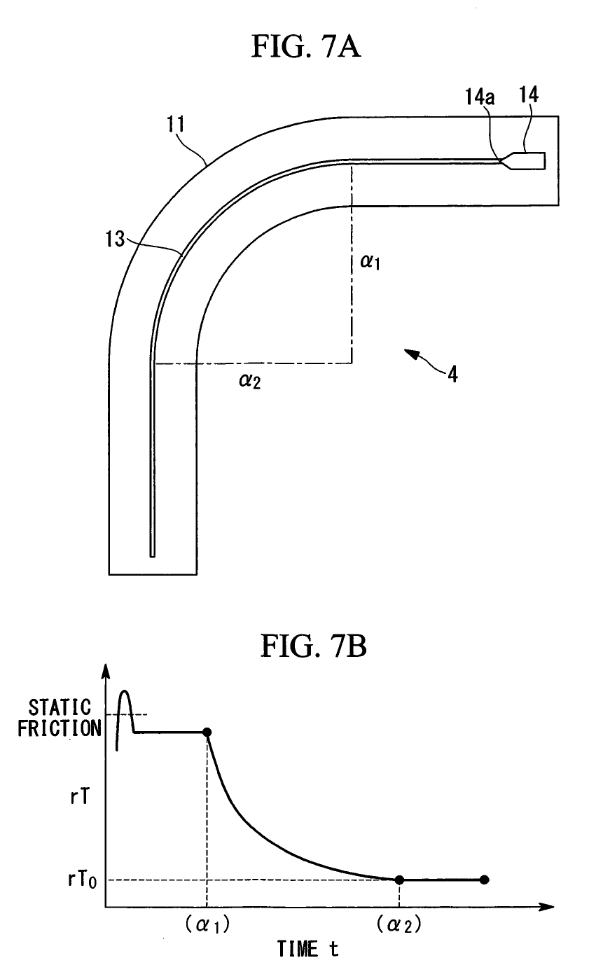

Also, in an example shown in FIG. 7A, since straight portions are provided at the both sides of the distal end side and the proximal end side of the arc-shaped portion of the cutter slot 13 in the cartridge 11, the motor 8 can be controlled to operate so that the torque does not change while the cutter 14 is located in the straight portions.

Also, a configuration in which the cutter 14, which is located at the distal end of the suturing section 4, is drawn to the proximal end side and cut tissue is described in this embodiment. In another embodiment, as shown in FIG. 8A, it is possible to employ a configuration in which the wire 15 is returned by a fixed pulley 19 provided at the distal end of the cartridge 11, and in witch tissue is cut when the cutter 14 is moved from the proximal end side to the distal end side.

In this configuration, torque rT according to the following expression (3) is generated by the motor 8.

rT=T₀×exp(μₛα)+p(θ, μₚ) (3)

In this expression, p(θ, μₚ) is friction loss force, θ is contact angle (approximately 180°) of the fixed pulley 19, and μₚ is friction coefficient between the wire 15 and the fixed pulley 19.

In this example, the wire 15 is returned and has approximately 230° of the contact angle α₁ when the cutter 14 is located at the proximal end side, and the wire 15 has approximately 115° of the contact angle α₂ when the cutter 14 is located at the distal end side.

Also, as shown in FIG. 8B, the cutter 14 may be fixed to the pulley 20, and the wire 15 may be returned again. With this configuration, it is possible to reduce the torque applied by the motor 8 to half of the torque calculated by the expression (3).

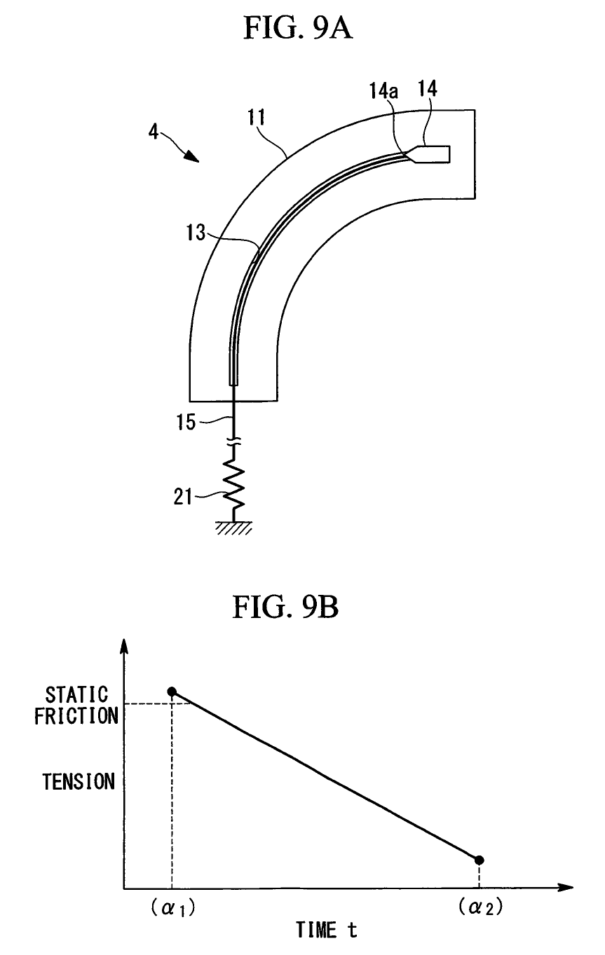

Also, this embodiment explains that the operational power is generated by the motor 8 and the controller 18 which controls the motor 8. In another embodiment, it is possible to generate tension which varies in response to the contact angle a as shown in FIG. 9B, using the spring 21 located at the proximal end side of the wire 15 as shown in FIG. 9A.

Further, it is possible to employ a flexible shaft as substitute for the wire 15.

Although the suturing device is illustrated as the tissue excision device 1 in the example, the present invention can be applied in a excision device which does not have a suturing device, or in an anastomosis device which conducts anastomosis with energy such as electric energy as substitute for the stapler.

The inventors have arrived at the following aspects of the invention.

An aspect of the present invention provides a tissue excision device comprising: a jaw which is disposed at a distal end of an insertion portion and which is used to sandwich tissue; a cutter which is movable along a track formed along the shape of the jaw; a drive unit which generates force to drive the cutter; and a force transmission member which connects the drive unit and the cutter and accommodated in an accommodation portion provided along the track, and which transmits the force generated by the drive unit to the cutter, wherein the drive unit is configured to generate greater force as reaction force that the force transmission member receives from the accommodation portion becomes greater.

According to this aspect, when the drive unit is operated while the insertion portion has been inserted in a body and tissue is sandwiched using the jaw provided at the distal end, the force generated by the drive unit is transmitted to the cutter via the force transmission member. Then, the tissue sandwiched by the jaw can be cut along the shape of the jaw by moving the cutter along the track which is formed along the shape of the jaw.

In this state, since the force transmission member is accommodated in the accommodation portion provided along the track, the cutter receives the reaction force by the friction between the member and the accommodation portion when the cutter moves. However, since the drive unit is configured so that the generated force becomes greater as the reaction force becomes greater, it is possible to reduce variation of the moving speed of the cutter even if the track of the cutter is curved. By this configuration, variation of blood circulation state in the tissue to be excised may not occur, and variation of the degree of tissue healing after the surgery can be reduced.

In the above-described aspect, it is preferable that the track comprises a portion which has a curvature.

With this configuration, it is possible to reduce variation of the moving speed when the cutter is moved along the track having a portion which has a curvature.

Also, in the above-described aspect, the force transmission member may be a flexible shaft or a wire.

With this configuration, the force transmission member as the flexible shaft or the wire causes friction on the internal wall of the accommodation portion, and therefore the longer the contact area is, the greater the force needed to drive the cutter is.

Also, in the above-described aspect, the drive unit may have a motor and a controller which controls torque to be generated by the motor.

With this configuration, it is possible to easily generate necessary torque in a configuration in which the force transmitted to the force transmission member becomes greater as the reaction force becomes greater.

Also, in the above-described aspect, the drive unit may be a spring which deforms the most at a position where the reactance force that the force transmission member receives from the accommodation portion becomes the greatest.

With this configuration, it becomes possible to reduce variation of the moving speed of the cutter without a complicated structure, but with a simple structure.

Also, in the above-described aspect, the force transmission member may have contact with an internal wall of the curved accommodation portion, and the controller controls the motor so that the force generated by the motor becomes greater as a contact angle between the force transmission member and the accommodation portion becomes greater.

Also, in the above-described aspect, the controller generates force in accordance with the following expression,

T=T₀×exp(μα)

wherein α is the contact angle between the force transmission member and the internal wall of the accommodation portion, μ is a friction coefficient, T0 is the force generated by the motor when α=0, T0 is the force generated by the motor.

The aforementioned aspects can achieve an advantageous effect of reducing variation of the moving speed of a cutter even when the track of the cutter is curved.

### REFERENCE SIGNS LIST

1 tissue excision device
2 insertion portion
8 motor
10 anvil (jaw)
11 cartridge (jaw)
13 cutter slit (accommodation portion, track)
14 cutter
15 wire (force transmission member)
18 controller (drive unit)
21 spring (drive unit)

1. A tissue excision device comprising:
a jaw disposed at a distal end of an insertion portion, wherein the jaw is configured to grasp a tissue;
a cutter configured to move along a track formed along the shape of the jaw;
a drive device configured to generate a force to drive the cutter; and
a force transmission member which connects the drive unit and the cutter, wherein the force transmission member is accommodated in an accommodation portion provided along the track, wherein the force transmission member transmits the force generated by the drive unit to the cutter,
wherein the drive device is configured to generate greater force as reaction force that the force transmission member receives from the accommodation portion becomes greater.

2. The tissue excision device according to claim 1, wherein the track comprises a portion which has a curvature.

3. The tissue excision device according to claim 1, wherein the force transmission member is a flexible shaft or a wire.

4. The tissue excision device according to claim 1, wherein the drive device comprises a motor and a controller which controls torque to be generated by the motor.

5. The tissue excision device according to claim 1, wherein the drive device comprises a spring, wherein the spring configured to deform the most at a position where the reactance force that the force transmission member receives from the accommodation portion becomes the greatest.

6. The tissue excision device according to claim 4, wherein the force transmission member is configured to contact with an internal wall of the curved accommodation portion,
wherein the controller is configured to control the motor so that the force generated by the motor becomes greater as a contact angle between the force transmission member and the accommodation portion becomes greater.

7. The tissue excision device according to claim 6, wherein the controller is configured to generate the force in accordance with the following expression,
T=T₀×exp(μα)
wherein α is the contact angle between the force transmission member and the internal wall of the accommodation portion, μ is a friction coefficient, T₀ is the force generated by the motor when α=0, T₀ is the force generated by the motor.
